(19) United States
(12) Patent Application Publication
(10) Pub. No.: US 2016/0228035 A1
(43) Pub. Date: Aug. 11, 2016

(54) NASO-ORAL DEVICE

(71) Applicant: FLEXICARE MEDICAL LTD., Mountain Ash Mid Glamorgan (GB)

(72) Inventors: Gassem POORMAND, Mountain Ash Mid Glamorgan (GB); Geraint WHITCOMBE, Mountain Ash Mid Glamorgan (GB)

(21) Appl. No.: 15/026,847

(22) PCT Filed: Oct. 3, 2014
(86) PCT No.: PCT/GB2014/052996

§ 371 Date: Apr. 1, 2016

(30) Foreign Application Priority Data
Oct. 3, 2013 (GB) 1317499.0

(51) Int. Cl. A61B 5/08; A61M 16/08; A61M 16/04; A61B 5/097; A61M 16/22

(52) U.S. Cl. CPC A61B 5/082; A61B 5/097; A61M 16/22; A61M 16/0493; A61M 16/085

(57) ABSTRACT

A naso-oral device (10) for use in medical procedures such as endoscopy includes a bite block (12) and a gas manifold or nasal cannula (14) which are releasably engageable. When engaged, gas supplied to the gas manifold passes, in use, (a) towards a patient's nostrils, through openings (58) in the gas manifold; and (b) into the patient's mouth via a passageway (28) through the bite block. Exhaled air is sampled for CO₂ monitoring via a coupling on the bite block and via nasal probes (74) on the gas manifold. The gas manifold can be detached from the bite block and still serve for both administering gas and sampling CO₂ in exhaled air.

The present invention relates to a naso-oral device for use in medical procedures such as endoscopy. The device facilitates the administration of gas (generally comprising oxygen) to a patient, and the sampling of exhaled gas.

EP 1 659 939 discloses an endoscopic mouthguard consisting of a bite block releasably coupled to a gas distribution manifold. The bite block has an annular body, like a napkin ring, with a peripheral flange at one end. In use the annular body is passed into a patient's mouth until the flange abuts around the mouth. A gas delivery passage is formed in the flange. It extends upwardly to an inlet opening and downwardly and inwardly to an outlet within the bore of the annular body. The gas distribution manifold is a small chamber, adapted to fit on top of the bite block, just under the patient's nose. It has a gas inlet tube. There are one or more upper outlet openings for passing gas to the patient's nostrils, and a back outlet for passing gas into the gas delivery passage in the bite block's flange, for passage through the annular body, into the patient's mouth.

In a commercial product embodying the patented invention (the TwinGuard (trademark) device of Trawax Pty Ltd), the bite block is adapted to be coupled to a CO₂ monitoring assembly for sampling CO₂ in exhaled air from the patient's nose and mouth. For this purpose, a nasal sampler is passed into one nostril.

The assembly is typically used while the patient is undergoing endoscopy, endoscopic apparatus being passed in and out through the annular body. After the procedure has finished, it may still be desired to administer gas to the patient. The bite block is uncoupled from the gas distribution manifold, and removed from the patient's mouth. The manifold remains adjacent the patient's nose and can continue to supply gas.

Broadly, the invention provides an endoscopic bite block system for oxygen delivery and carbon dioxide monitoring, the bite block system comprising:

a bite block for positioning at least partially within a human mouth, the bite block comprising an endoscope channel sized to enable an endoscope to pass therethrough; and

a nasal cannula detachably connected to the bite block, the nasal cannula comprising:

a delivery portion comprising a nasal outlet port to provide oxygen to a human nostril and an oral outlet port to provide oxygen to a human mouth, the oral outlet port positioned opposite the nasal outlet port; and

a monitoring portion comprising a nasal tube terminating at a nasal in port to sample air expelled from a human nostril. It may include:

a nasal sampling line in fluid communication with a main sampling line and the nasal tube of the monitoring portion of the nasal cannula; and

an oral sampling line in fluid communication with the main sampling line and an oral sampling channel of the bite block, wherein the oral sampling line comprises a valve. The oral sampling line may comprise a connector enabling the oral sampling line to be separated into at least two pieces. Desirably the valve is part of the connector, and the valve automatically closes upon separating the oral sampling line into the at least two pieces. The connector may comprise a Luer connector.

There is preferably a main delivery line connected to an inlet port of the delivery portion to provide oxygen to the delivery portion, the main delivery line being monolithically attached to the main sampling line along a length of the main delivery and sampling lines.

The delivery portion and monitoring portion may form a unitary structure.

The delivery portion may have a second nasal outlet port, the nasal outlet ports being separated by a distance that enables the outlet ports to each be positioned beneath a different nostril.

The monitoring portion may include a second nasal tube terminating at a second nasal inlet port to sample air expelled from a second human nostril, the nasal tubes separated by a distance that enables the nasal tubes to each be positioned within a different nostril.

The oral outlet port may be positioned at an end of an oral delivery channel extending in a direction opposite the nasal outlet port.

The nasal cannula may be detachably connected to the bite block by the oral delivery channel being positioned within a cavity of the bite block, the cavity of the bite block in fluid communication with at least one oral outlet channel of the bite block, the at least one oral outlet channel positioned within the endoscope channel.

The bite block may have an outer surface extending around the endoscope channel, the outer surface having a first durometer (hardness); and

an elastomeric layer overmolded on at least a portion of the outer surface, the elastomeric layer having a second durometer, which is softer than the first durometer.

The nasal and oral sampling lines preferably comprise tubing having a channeled inner lumen to resist kinks and occlusion.

According to a preferred class of embodiment of the present invention there is provided a naso-oral device comprising an annular bite block and a gas manifold, wherein the gas manifold is releasably engageable with the bite block to communicate with a gas supply path through the block for supplying gas to a patient's mouth; the gas manifold also including a nasal supply passage for supplying gas to a patient's nose; the device also having a first CO₂ monitoring line connected to a central region of the bite block for monitoring orally exhaled gas; and a second CO₂ monitoring line connected to the gas manifold for monitoring nasally exhaled gas; wherein the gas manifold can be disconnected from the bite block and used to supply gas and to monitor exhaled gas.

An embodiment of the invention will be described with reference to the accompanying drawings in which:

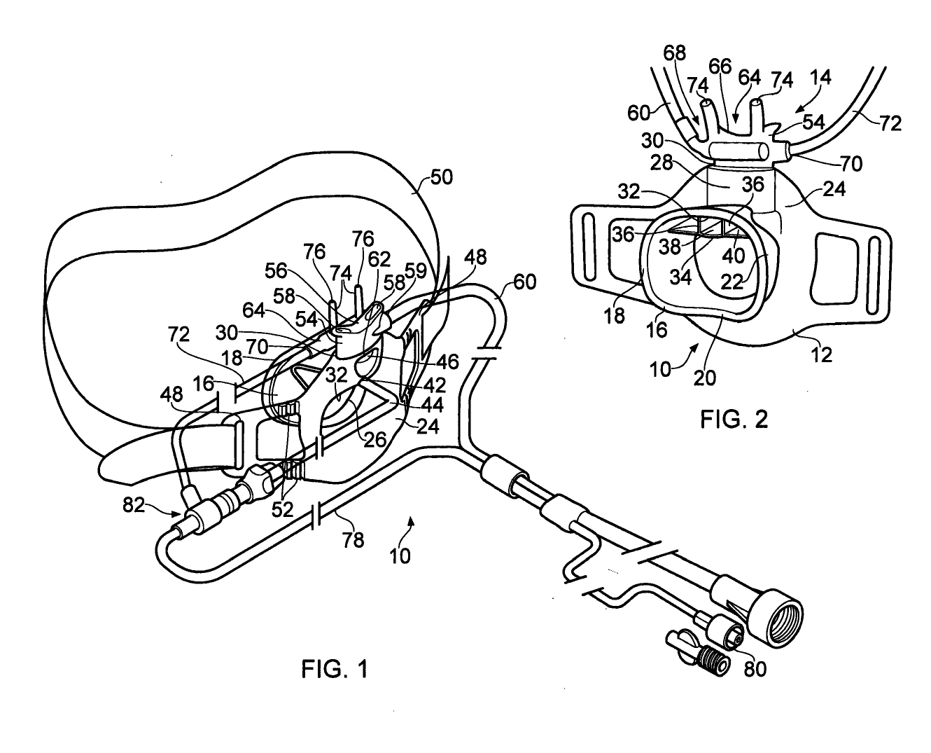

FIG. 1 is a front perspective view of a naso-oral assembly comprising a device embodying the invention; and

FIG. 2 is a rear perspective view showing the bite block and nasal cannula assembled together.

The illustrated assembly 10 includes a bite block 12 and a gas manifold (or nasal cannula) 14. The bite block 12 comprises a unitary plastics moulding. A tubular body portion 16 has a rounded rectangular cross-section and slightly increases in diameter from front to rear. At the rear it terminates with a peripheral rim 18. At the bottom there is a recessed area 20 for accommodating a patient's tongue. The external surface of the body portion 16 has an overmolding 22 of softer, rubbery plastic, engageable more comfortably and safely by a patient's teeth.

At the front of the body portion 16 it is surrounded by a peripheral flange portion 24. There is a smooth transition from the front of the flange portion 24 to the interior of the tubular body portion 16, for guiding objects that are to be passed in through the body portion 16. As viewed from the front, the opening 26 of the body portion 16 is wide and uncluttered.

The central upper region of the flange portion 24 defines a gas passage 28 extending downwardly from a laterally elongate upper opening 30. At the bottom, the passage 28 turns inwardly, the final section being within the tubular body portion 16, defined between the upper surface 32 of the body portion and a U-shaped projecting flange 34 that extends parallel to that surface a short distance below it, terminating a little in front of the recessed area 20. The flange 34 is connected to the upper surface 32 by two webs 36 that define a central conduit 38 and two side passages 40. The central conduit 38 leads to a front opening 42 where a monitor tube 44 is connected. The side passages 40 join the main gas passage 28. In the front wall of the gas passage 28, there is a small opening 46.

A-shaped limbs 48 extend on each side of the peripheral flange portion 24 for attachment of a strap 50. Adjacent to the flange portion 24, there are several grooves 52 constituting living hinges, to facilitate flexing of the limbs 48.

The gas manifold or nasal cannula 14 has a front portion which is a gas conduit 54. It is a tubular body, open at the bottom. Its top surface, provided by a top plate portion 56, is laterally arcuate, rising from the centre. The top plate portion 56 has an opening 58 at each lateral end. The spacing of the openings 58 is similar to the typical spacing of human nostrils. There are short internal partitions such that the openings 58 lead into respective tubular conduits extending about half way down the body 54. A lateral port 59 opens into one of the tubular conduits and is coupled to an oxygen supply line 60. The front face of the body 54 has a raised nib 62. The body is formed of flexible plastics material, dimensioned so that it can be pushed into the gas passage 28 in the bite block 12, through the opening, resiliently deforming the body 54 to allow passage of the nib 62 until this can project through the small opening 46 in the front wall of the gas passage 28. The gas manifold 14 is then securely, but releasably, coupled to the bite block 12, in a precise location.

The gas manifold 14 also has a rear portion which is a monitoring chamber 64. This has a laterally-extending tubular body 66, closed at one end 68 and having a port 70 at the other end, coupled to a CO₂ monitoring line 72.

Two narrow tubular extensions 74 rise up from the body 66 for about 1 cm, curving slightly to the rear. They terminate in smooth apertures 76. They are very soft and flexible, so they can be passed into a patent's nostrils without risk of injury or discomfort.

A main monitoring line 78 extends from a connector 80 (for connection to a gas monitor) to a branch unit 82 from which the two monitoring lines 44,72 extend. The branch unit 82 is designed so that the line 44 leading to the front opening 42 of the bite block 12 can be uncoupled, and the unit 82 seals the inlet from which the line 44 was uncoupled. Communication along the other monitoring line 72 is unaffected.

In use, the assembly as shown in FIG. 1 is coupled to an oxygen source and a CO₂ monitor. The body portion 16 of the bite block 12 is passed into a patient's mouth, and the tubular extensions 74 are carefully positioned to extend into the patient's nostrils. The assembly is held in place by means of the straps. The patient can now breathe in supplied oxygen by mouth and/or through the nostrils. The CO₂ content of gas exhaled through either mouth or nostrils can be monitored. An endoscopic procedure can be carried out, endoscopy probes etc. being fed through the tubular body portion 16 of the bite block.

After the procedure has finished, the bite block 12 is removed. The nasal cannula 14 is separated from the bite block 12. The bite block's monitoring line 44 is separated from the branch unit 82. The nasal cannula 14 is then re-mounted adjacent the patient's nose so it can continue to supply oxygen (to the nose via the tubular extensions 74, and to the mouth, through the opening at the bottom of the tubular body 66), and to monitor exhaled gas.

1. A naso-oral device comprising an annular bite block and a gas manifold, wherein the gas manifold is releasably engageable with the bite block to communicate with a gas supply path through the block for supplying gas to a patient's mouth; the gas manifold also including a nasal supply passage supplying gas to a patient's nose; the device also having a first CO₂ monitoring line connected to a central region of the bite block for monitoring orally exhaled gas; and a second CO₂ monitoring line connected to the gas manifold for monitoring nasally exhaled gas; wherein the gas manifold can be disconnected from the bite block and used to supply gas and to monitor exhaled gas.

2. The naso-oral device of claim 1 wherein the second CO₂ monitoring line communicates with a pair of monitoring probes provided on the manifold and spaced so as to sample gas from respective nostrils.

3. The naso-oral device of claim 1 wherein the first CO₂ monitoring line communicates with a sampling conduit provided by the bite block, the bite block defining an endoscope channel extending from the front to the rear, and wherein the sampling conduits has a sampling inlet openings that is at or adjacent the rear of the device, in a central upper region of the endoscopic channel.

4. The naso-oral device according to claim 1 wherein the external surface of the bite block has an overmolding of relatively soft rubbery material for engagement by a patient's teeth.

5. The naso-oral device of claim 1 wherein the first CO₂ sampling line comprises a valve.

6. The naso-oral device of claim 5, wherein the first CO₂ sampling line comprises a connector enabling the line to be separated into at least two pieces.

7. The naso-oral device of claim 6, wherein the valve is part of the connector, and the valve automatically closes upon separating the oral sampling line into the at least two pieces.

8. The naso-oral device of claim 1, wherein the gas manifold has a locking protrusion shaped to fit within a mating slot of the bite block, wherein the nasal cannula is lockable into position with the bite block only in the correct orientation.
